(19) United States
(12) Patent Application Publication
(10) Pub. No.: US 2008/0071160 A1
(43) Pub. Date: Mar. 20, 2008

(54) Displaying A Tracheobronchial Tree

(75) Inventors: Rafael Wiemker, Kisdorf (DE); Thomas Blaffert, Hamburg (DE); Thomas Buelow, Grosshansdorf (DE); Steffen Renisch, Hamburg (DE); Cristian Lorenz, Hamburg (DE); Hans-Aloys Wischmann, Henstedt-Ulzburg (DE)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N.V., Eindhoven (NL)

(21) Appl. No.: 11/570,517

(22) PCT Filed: Jun. 15, 2005
(86) PCT No.: PCT/IB05/51968

§ 371 Date: Dec. 13, 2006

(30) Foreign Application Priority Data
Jun. 22, 2004 (EP) 04102883.8

(51) Int. Cl. A61B 5/04

(52) U.S. Cl. 600/407

(57) ABSTRACT

The invention relates to automatically segmenting and displaying the tracheobronchial tree (400) and displaying clinical values (404) related to the segmented tracheobronchial tree (400).

The invention relates to a method of displaying a tracheobronchial tree of a body.

The invention further relates to a system for displaying a tracheobronchial tree of a body.

The invention further relates to an image acquisition device comprising such a system.

The invention further relates to an image workstation comprising such a system.

The invention farther relates to a computer program product designed to perform such a method.

The invention farther relates to an information carrier comprising such a computer program product.

An embodiment of such a method and system is disclosed in U.S. Pat. No. 6,272,366. Here a method and system are provided for effecting interactive three-dimensional renderings of selected body organs for purposes of medical observation and diagnosis. A series of Computer Tomography (CT) images of the selected body organs are acquired. The series of CT images is stacked to form a three-dimensional volume file. To facilitate interactive three-dimensional rendering, the three-dimensional volume file may be subjected to an optional dataset reduction procedure to reduce pixel resolution and/or to divide the three-dimensional volume file into selected sub-volumes. From a selected volume or sub-volume, the image of a selected body organ is segmented or isolated. Image segmentation may be effected by various techniques. For example, an image slice through the three-dimensional volume file may be subjected to a thresholding process in which a physical property of the two-dimensional image slice, such as x-ray attenuation, may be used to established a particular threshold range, such as a range of x-ray attenuation values, that corresponds to the organ of interest. After an appropriate threshold range is determined, the entire three-dimensional volume file is then thresholded to segment the organ of interest. For example, in order to segment the colon, a threshold range corresponding to the air column within the colon could be selected to isolate the inner wall of the colon.

An alternative segmentation technique may be employed in which a region growing technique is used to isolate the air column within the colon. Using the region growing technique, a “seed” is planted by selecting a data point or voxel within the air column of the colon. Neighboring voxels are progressively tested for compliance with a selected acceptance criteria, such as x-ray attenuation values falling within a selected threshold range representing air. As such, the seed region continues to expand or grow until the entire air column within the lumen of the colon is filed.

A surface, or isosurface, of the air column representing the colon is then produced. A wireframe model of the isosurface is then generated using a selected image processing technique such as a marching cubes algorithm. From the wireframe model of the colon, a three-dimensional interactive rendering is produced that enables the user to rapidly view a series of three-dimensional images of the lumen of the colon for purpose of detection of pathological conditions.

Hence, the user must manually measure from these images, the relevant clinical parameters that are for example relevant in determining the diagnosis and treatment of asthma, bronchiectasis, emphysema, and other pulmonary diseases.

It is an object of the invention to provide a method according to the opening paragraph that allows assessment of the tracheobronchial tree in an improved way. To achieve this object, the method comprises automatically segmenting the tracheobronchial tree from a three-dimensional image set of a body; automatically determining a quantitative measurement based upon the tracheobronchial tree; displaying the quantitative measurement in addition to the displayed tracheobronchial tree. By providing an automatic segmentation of the tracheobronchial tree, the segmentation can be performed unsupervised. Thereby allowing the segmentation to be performed automatically upon, before or after loading other information of the body such as other images, demographic data etc. The body can either be a patient or an animal. By displaying the tracheobronchial tree automatically, the assessment of this tree can be done more easily. Further, by automatically determining a quantitative measurement, this quantitative measurement can be performed upon, before or after loading other information of the body too. Advantageously, those quantitative measurements are performed and displayed that have a clinical value and allow for faster assessment of the tree.

In a further embodiment of the method, the method comprises: extracting centerlines of trachea, bronchi, and/or smaller airways based upon the tracheobronchial tree; determining branching points of the tracheobronchial tree based upon the extracted centrelines. By determining the centerlines of the trachea, bronchi and/or smaller airways and determining branching points of the tree, these can be used as a basis to determine clinical values along the complete or part of the tracheobronchial tree. Advantageously, a bronchial segment or sub-segment starts and ends between branching points and branching points can be used to display the bronchial segment or sub-segment limited by branching points.

In a further embodiment of the method, the method comprises determining for at least one centerline point at least one of a bronchial lumen, a lumen diameter, an inner radius from the centerline point to an inner bronchial wall, an inner diameter based upon the inner radius, an outer radius from the centerline point to an outer bronchial wall, an outer diameter based upon the outer radius, an artery radius of an accompanying artery, an artery diameter based upon the artery radius, wherein the centerline point comprises a point on a centerline of the extracted centerlines. By determining at least one of these values, these values can be used to derive other clinical values from that enable assessment of the body's airway structure.

In a further embodiment of the method, the method comprises determining for at least one branching point at least one of a first difference between the outer radius and the inner radius as a function of the lumen diameter, a second difference between the inner bronchial diameter and the artery diameter as a function of the lumen diameter. By determining at least one of these values, these values too can be used to derive other clinical values from that enable assessment of the body's airway structure.

In a further embodiment of the method, the method comprises displaying an indicator indicating a position in the tracheobronchial tree corresponding to the quantitative measurement. By indicating the a position within the tree corresponding to a quantitative measurement, a user can easy see which quantitative measurement corresponds to what position within the tree.

In a further embodiment of the method, the indicator indicates an anomaly within the tracheobronchial tree. By using the indicator to indicate anomalies, the user can better assess the body's airway structure. Further the attention of the user is drawn to anomalies.

In a further embodiment of the method, an image acquisition device designed to reconstruct a volumetric image set acquires the three-dimensional image set. Such a volumetric image set can for example be acquired by a CT apparatus and by using a CT-image set, the properties of the CT-image set can be used to determine the tracheobronchial tree. Other image acquisition devices can be used too, for example an MR scanner, a 3-Dimensional Rotational Angiography (3D-RA) scanner, Positron Emission Tomography (PET) scanner, or Single Photon Emission Computed Tomography (SPECT) scanner.

In a further embodiment of the method, the tracheobronchial tree is displayed partially. By partially displaying the tracheobronchial tree, only the left or the right or the tree per lung lobe can be displayed. Thereby allowing the user to focus more upon a part of the body's airway structure.

In a further embodiment of the method, a user can manipulate the automatic segmentation and/or the displayed tracheobronchial tree. Discrepancies between the displayed tree and an expected tree can be overcome by allowing the user some manipulation.

It is an object of the invention to provide a system according to the opening paragraph that allows assessment of the tracheobronchial tree in an improved way. To achieve this object, the system for displaying a tracheobronchial tree comprises segmentation means for automatically segmenting the tracheobronchial tree from a three-dimensional image set of a body.

Embodiments of the system are described in claims 11 to 16.

These and other aspects of the invention will be apparent from and elucidated

with reference to the embodiments described hereinafter as illustrated by the following Figures.

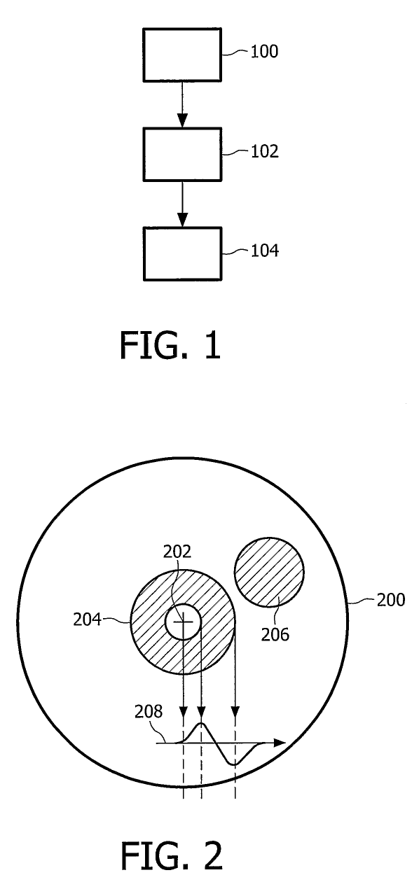

FIG. 1 illustrates the main steps of method according to the invention;

FIG. 2 illustrates how the inner and outer bronchial wall is measured;

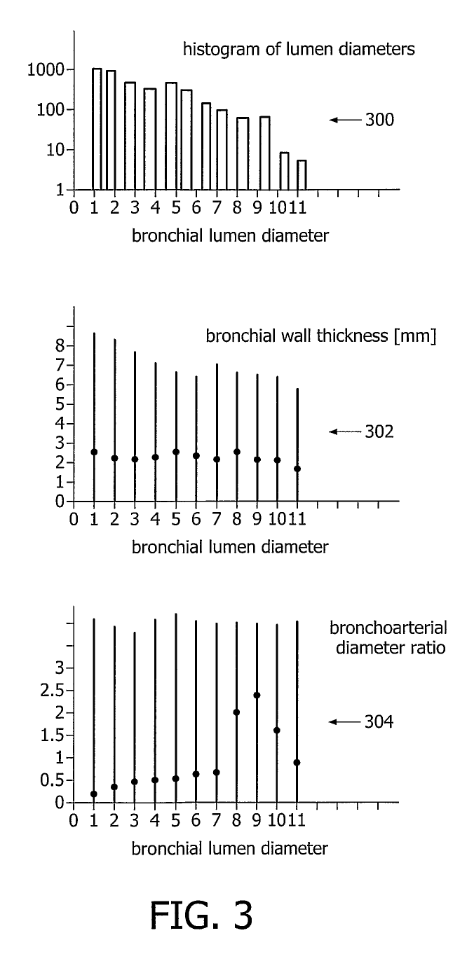

FIG. 3 illustrates examples of representing clinical parameters graphically;

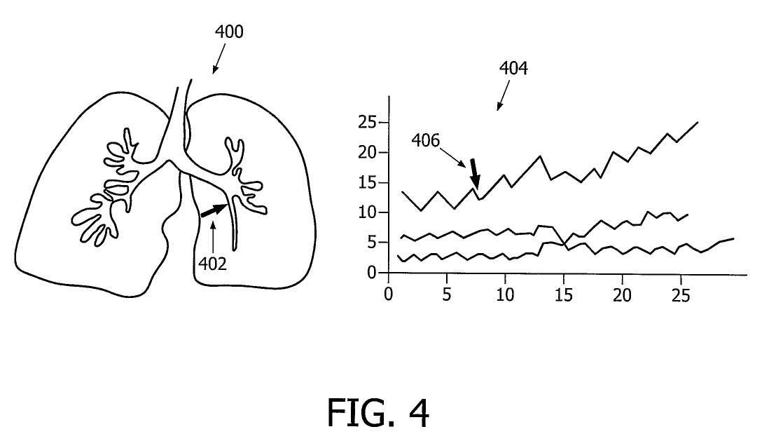

FIG. 4 illustrates the visualization of the tracheobronchial tree together with a graph comprising clinical values;

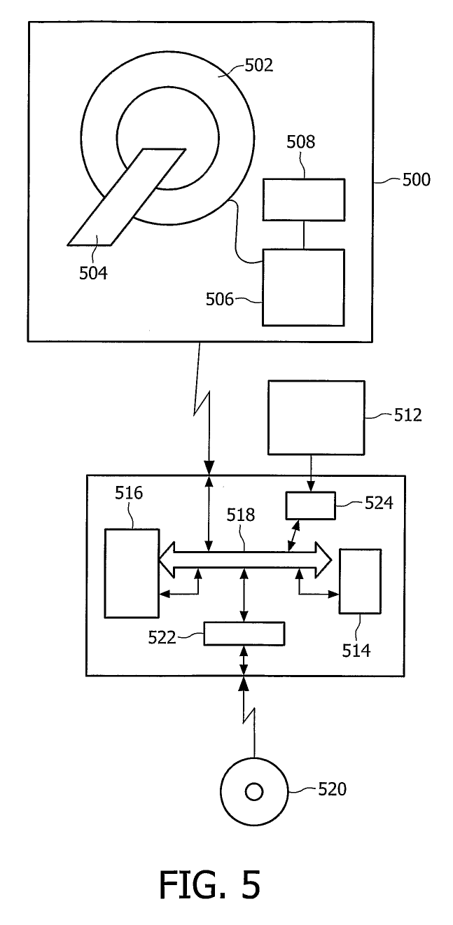

FIG. 5 illustrates a medical apparatus according to the invention in a schematic way.

The human lung consists of two major parts, the left lung and the right lung. There are three lobes in the right lung, which are separated by the so-called major fissure and minor fissure. The left lung shows a slightly different structure. Because there is no defined minor fissure, it consists of only two lobes, whereby the part that anatomically corresponds to the right middle lobe is merged with the upper lobe. Each lobe is again divided into two or more lung segments of which ten exist for each side of the lung. These segments are supplied by a complex system of branching trees that conduct blood and air into the distal regions where the gas exchange takes place. The bronchial tree has a pipe structure that is filled with air. It starts at the trachea and extends into the distal regions repeatedly splitting into smaller and smaller branches. In the human lung, the splitting occurs usually in bifurcations, e.g. the parent branch splits up into two child branches, but trifurcations also exist. The general tendency for child branches is that they decrease in diameter and length although this might be different in individual cases. Siblings don't necessarily have the same diameter. The bronchi are classified into lobar bronchi that supply the lobes, segmental bronchi, that supplying the individual segments, and sub-segmental bronchi. The bronchial wall surrounds the air-filled lumen of the bronchi. The thickness of this wall is correlated to the diameter of the segment in the sense that it gets thinner for smaller diameters. High-resolution multi-slice CT reveals bronchi segments in the 6^{th} branching generation and higher which have diameters in the mm range. For diagnosis and treatment of asthmatic and emphysematic patients, the bronchial lumen, bronchial wall thickness, and the ratio of inner bronchial to accompanying arterial diameter are parameters which are used in clinical practice in order to detect and quantify airway narrowing, bronchial dilation, bronchial wall thickening, bronchiectasis, hyperresponsiveness, etc.

FIG. 1 illustrates the main steps of method according to the invention. Three main steps 100, 102 and 104 can be distinguished in the method according to the invention.

Within the first step 100, the tracheobronchial tree is automatically segmented. The segmentation starts with loading a three-dimensional image set (3D-image set) of a thorax. The 3D-image set is preferably acquired with a high resolution CT scanner, such as a multi-array CT scanner. The high resolution refers to a slice thickness of about 1.0-1.3 mm or less. Other 3D-image sets that are acquired by a scanner that can produce such a high resolution image set, for example an MR scanner, a 3D-RA scanner, PET scanner, or SPECT scanner, etc. can be used too. Further the 3-D image set can be acquired with and without contrast agent, cardiac or respiratory gating. If a CT 3D-image set is used, the lung and trachea area can be segmented out of the overall 3D-image set of the thorax by setting a Hounsfield threshold, f.e. at −500 HU and identifying all 3D-connected voxels below the Hounsfield threshold. Then, the lung and trachea area is identified as the largest component of 3D-connected voxels that is not touching the image boundaries. Next, the trachea must be determined. For this purpose, the first voxel in a direction perpendicular to the plane of the slices, i.e the z-direction, that belongs to the lung and trachea area is found, and also the last voxel in this direction, since the scan-direction can be head-to-feet or feet-to-head. Of these two voxel positions, the one is chosen which is more central in the plane of the slices, i.e. the xy-direction. If the image set comprises descriptive data indicating the scanning direction, this descriptive data can be used to determine the trachea.

Within the next step 102, the centerlines of the trachea, segmental bronchi and smaller airways, i.e. the sub-segmental bronchi are extracted. Further the branching points of the tree structure are determined. This step is based on a front propagation approach which detects “leakages” into the parenchymal tissue, see also T. Schlathölter, C. Lorenz, I. C. Carlsen, S. Renisch, T. Deschamps, Simultaneous Segmentation and Tree Reconstruction of the Airways for Virtual Bronchoscopy.

Proceedings SPIE Medical Imaging 2002, SPIE vol. 4684, part 1, pp. 103-113. Here, the front propagation method is used in conjunction with an anatomical model of the tracheobronchial tree. The front propagation method is a type of region growing technique that uses a concept motivated from physical wave-front propagation and that is based on the physical principle of least action. The front propagation method uses a fast marching algorithm, for example as described in T. Deschamps, L. D. Cohen, Minimal Paths in 3D images and application to virtual endoscopy, Lecture Notes in Computer Science: Computer Vision—ECCV 2000; 1843:543-557.

The front propagation equation used is of the type:

|∇T|F=1 (1)

where F(x) is the speed function of the front and T(x) denotes the time value when the front reaches the point x. A stepwise constant speed function is used of the following form:

$$F ( x ) = \{ \begin{matrix}1 & for I ( x ) \leq t \\ 0 & for I ( x ) > t\end{matrix} \qquad ( 3 )$$

with t being a threshold value just above the bronchial lumen, and I(x) denoting a gray value at the point x.

The front propagation method keeps a list of branches that have to be grown. This list is initialized with the trachea. After initialization the algorithm loops over a sequence of growing, branch detection, and branch validation.

Growing: consecutively, one branch is taken from the list and is grown according to the modified fast marching algorithm described above. Each branch keeps a reference to its initial radius (rᵢ) and compares this after every grow step to the actual radius. When the current branch approaches a bifurcation, the actual radius increases and finally exceeds the initial radius times a multiplication factor α (e.g. α=1.1).

Branch detection: when the actual radius exceeds (α*rᵢ, a check for branching is performed. Using α, the execution of the computationally expensive connectivity checking process can be reduced. In case no branching is detected, α is increased about 0.1 and the grow process is continued. In the case of branching, the validity of the current branch is checked. This process is responsible for the detection of leakage. When a branch is detected, this is stored for example in a linked list structure that represents the branching points of the tree structure.

Branch validation: After branching occurred, the validity of the parent branch B of the branches Bᵢ can be verified. Validation is responsible for rejecting branches that most probably represent leaked regions. Two criteria: radius and connectivity are used for the validation.

Radius: Since the grid point distribution of each branch is known from the segmentation result, it is possible to calculate its covariance matrix. Using a cylindrical model of the tracheobronchial tree, it is possible to estimate an average radius of the branch using the lowest two eigenvalues (EV) of the covariance matrix:

$${r}_{s} = \frac{{EV}_{2} + {EV}_{3}}{2} .$$

Since generally the radius is decreasing with increasing branch order a radius smaller than β*rₘᵢₙ (with rₘᵢₙ being the smallest radius of all ancestors) indicates leakage. β is chosen to be greater than 1 to provide a safety margin to the internal variability of the radius of the branches.

Connectivity: By checking the neighbor voxels of all surface voxels of a branch B, one can find the number of branches, which are in the direct vicinity of B. If one compares the number of different branches in the direct neighborhood with the maximum number of allowed branches (γ) one can detect leakages. γ should be set to an integer number greater than three. Three neighbors is the usual case since a branch usually has a parent and two children. Three or more children are also possible, thus this parameter should be chosen carefully, not too low and not too large (e.g. γ=5).

For valid branches, the unconnected regions of the front are used to initialize new branches, which are stored in the branch list; for invalid branches they are discarded. Thus during the growth, each “front voxel” belongs to one of several 3D-connected growth fronts. If one of these fronts becomes too large, then it is considered “leakage” and this front is frozen, and only voxels from other fronts are propagated.

The region growing of the tracheobronchial tree can be repeated several times, starting with a high Hounsfield thresholds (e.g. −800 HU), and then descending to lower thresholds (e.g. down to −900 HU in steps of 20 HU), where the resulting voxels from each iteration are taken as seeds for the next iteration.

The centerlines can be determined by computing a distance map for the segmented volume of the bronchial tree, giving the distance for each voxel to the nearest non-bronchi voxel. The distance can be derived from the radius. Such a non-bronchi voxel is part of the surrounding lung parenchyma tissue. All bronchial centerlines can be written into a table with the original trachea seed-point as the endpoint and the most distal point as the start-point. Then all bronchi can be traced for left and right lung separately, and measurements of the clinical parameters below can be taken at each trace point.

Within the final step 104, clinical parameters are determined for the segmented tracheobronchial tree and these are displayed preferably together with the segmented tree. At each point along all bronchial centerlines, the bronchial lumen that is equal to two times the radius of the inner bronchial wall, the radius to the outer bronchial wall, and the thickness of the accompanying artery is measured. Thus, the mean wall thickness and the mean ratio of inner bronchial to accompanying arterial diameter can be given as a function of lumen diameter. The mean wall thickness is defined as the difference between outer and inner bronchi radius.

FIG. 2 illustrates how the inner and outer bronchial wall is measured. Here, 200 indicates a three-dimensional sphere, 202 indicates a point on the bronchial centerline, 204 indicates a bronchus, 206 indicates an accompanying artery, 208 indicates a graph of the mean radial derivative of the bronchi. The three-dimensional sphere 200 of radius r is placed around a given point 202 on the bronchial centerline. On the surface of the sphere sample the radial derivative ∂HU/∂r of the Hounsfield values is sampled. The radial derivatives are computed as discrete differences ΔHU/Δr with a base length Δr equal to the in-slice voxel spacing for example 0.6-0.8 mm. The discrete differences AHU are not computed on a voxel grid, but rather between continuous coordinate positions, the Hounsfield values of which are estimated by tri-linear interpolation. Then this sphere is expanded with radius steps dr of 0.25 mm. The inner bronchial wall radius is determined by taking the radius at which the mean radial derivative reaches a maximum, see graph 208. The outer wall radius is determined by taking the radius at which the mean radial derivative reaches a minimum, see graph 208. Moreover, the central position is also slightly varied into x,y and z direction until the sphere with maximum mean radial derivative is found. As all radial derivatives are computed with tri-linear interpolation of the Hounsfield values, the radius and position of the best fitting sphere can be computed to sub-voxel accuracy. Advantages of this way of radius estimation are that

it is not dependent on certain Hounsfield thresholds;

it works also on only partially closed bronchial walls;

it yields three-dimensional and subvoxel accuracies;

there is a clear criterion when to accept a measurement point: if the mean radial derivative curve shows a pronounced minimum following a pronounced maximum;

the measurement does not depend on the estimation of the local airway axis;

the same measurement principle can be applied for inner and outer airway wall as well as for artery diameters.

The search for an accompanying artery is conducted in a sphere of three times the radius of the outer bronchial wall around the center point 202. Within this search sphere the largest structure with vessel-morphology is identified. The measurements of the radii as well as the search for the accompanying artery can also be done in a two-dimensional disk perpendicular to the centerline of the airway.

FIG. 3 illustrates examples of representing clinical parameters graphically. Histogram 300 illustrates a histogram of lumen diameters with a bin size of 0.5 mm. Graph 302 gives a graph representing the bronchial wall thickness, and graph 304 gives a graph representing the bronchoarterial diameter ratio. Depending on the depth of successful airway segmentation, the algorithm can determine a plurality of suitable measurement points for each thorax dataset for determination of the bronchial lumen diameter and the bronchial wall thickness, for example between 1000 and 5000 suitable measurement points. Due to the tree-structure of the airways, the frequency of lumen diameters can be given in a logarithmic scale. Then mean and standard deviation for wall thickness and bronchoarterial diameter ratio can be computed.

The clinical parameters can be given for left and right lung separately or even per lobe. For example, a curve for each parameter for the principal longest bronchial centerlines can be given or a scatter plot with the clinical parameters as a function of distance to carina, which is a branching point in trachea, or as a function of bronchial lumen. Further, more different histograms of clinical parameters can be calculated. For example: the percentage of the length of the tracheobronchial tree which exhibits a certain lumen, wall thickness, the mean wall thickness and artery diameter ratio for bronchi pieces split up for different lumen ranges, etc.

FIG. 4 illustrates the visualization of the tracheobronchial tree together with a graph comprising clinical values. The visualization of the tracheobronchial tree 400 comprises a pointer 402. The graph of clinical values 404 as previously describes comprises an other pointer 406. The pointers 402 and 404 indicate corresponding positions between a value of the graph and the position within the tree that this value corresponds with. By using these pointers a user can easily navigate over the tree by manipulating the pointer 402 and see the corresponding values indicated by pointer 406. The user can also manipulate pointer 406 thereby causing the pointer 402 to indicate the corresponding position within the tree. As a further aid to the user, the positions of parametric anomalies, such as such as bronchial lumen obstructions, lumen dilations, bronchial wall thickenings can be marked graphically in the original 3D image set, and in a coronal and sagittal overview image. Further different anomalies can be represented by a different shape, color, etc of the pointer. Therefore, the method according to the invention provides a user with a tracheobronchial segmentation method that displays the result of the segmentation together with clinical values that requires minimal user interaction. Preferably the method does not require user interaction at all and can thus be performed for example as a background job running on a suitably programmed computer. Thereby saving time for the user as he his presence is not required to segment the tree or calculate the clinical values. This background job can for example be started immediately after acquisition of the image set. Automatic segmentation and calculation of clinical values leads to reproducible results and can therefore contribute to a better evaluation of clinical images. In order to give the user some control of the segmenting process and the calculation of the clinical values, the method can provide steps to the user in which the user can manipulate basic parameters that are used in the segmentation process, such as the Hounsfield value, or the branches of the tracheobronchial tree that should be segmented. The method can further provide steps to the user in which he can select for example which clinical values to calculate and display. Further the user can view the original image set in different orthogonal views together or without the segmented tree and the clinical values. The clinical values can be displayed as graphs, as numbers, and other visualization techniques that are suitable for the intended purpose.

FIG. 5 illustrates a medical apparatus 500 according to the invention in a schematic way. The medical apparatus 500 is an CT acquisition device that comprises a multi-array CT gantry 502 and a patient table 504 that can be positioned within the gantry 502. The patient table 504 supports a patient during acquisition of coarse image data of the patient. The coarse image data is applied to a microcomputer 506, which reconstructs volumetric image data of the coarse image data. The computer is programmed in such a manner that in conformity with the invention it calculates a segmented tree of the tracheobronchial tree and corresponding clinical values, said tree and values being displayed on the display unit 508 of the computer. Alternatively, the reconstructed volumetric image data can be transferred to an image processing system 510 for processing the data according to the method of the invention. This image processing system 510 may be a suitably programmed computer of a workstation. The workstation is connected to a screen 512. The system 510 further comprises a microprocessor 514, a general purpose memory 516 like random access memory (RAM) and a further memory 524 that are being communicatively connected to each other through a software bus 518. The memory 516 comprises computer readable software code designed to perform the method according to the invention as previously described. The memory 524 is a display buffer that is designed to comprise the segmented tracheobronchial tree, the graphs of clinical values and the original image set as previously described. The contents of this display buffer is displayed at the display device 512. It is further possible to download the computer readable software from a storage device like a compact disk (CD) 520, digital versatile disk (DVD) etc. or to download the computer readable software as such from the Internet into the memory of the workstation. Therefore, the workstation comprises a suitable storage reading device 522, like a CD-drive, that can read the software from the storage device. This CD-drive is then operatively connected to the software bus too. Within the previous example, the invention is described with reference to an CT acquisition device. However, the invention is not limited to a CT acquisition device, but extends to all imaging devices capable of reproducing volumetric image data, like for example 3D-RA, MR, PET, SPECT, etc.

The order in the described embodiments of the method of the current invention is not mandatory, a person skilled in the art may change the order of steps or perform steps concurrently using threading models, multi-processor systems or multiple processes without departing from the concept as intended by the current invention.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. The invention can be implemented by means of hardware comprising several distinct elements, and by means of a suitably programmed computer. In the system claims enumerating several means, several of these means can be embodied by one and the same item of computer readable software or hardware. The mere fact that certain measures are recited in mutually different dependent claims does not indicate that a combination of these measures cannot be used to advantage.

1. Method of displaying a tracheobronchial tree comprising automatically segmenting the tracheobronchial tree from a three-dimensional image set of a body;
automatically determining a quantitative measurement based upon the tracheobronchial tree;
displaying the quantitative measurement in addition to the displayed tracheobronchial tree.

2. Method according to claim 1, comprising:
extracting centerlines of trachea, bronchi, and/or smaller airways based upon the tracheobronchial tree;
determining branching points of the tracheobronchial tree based upon the extracted centerlines.

3. Method according to claim 2, comprising determining for at least one centerline point at least one of
a bronchial lumen,
a lumen diameter,
an inner radius from the centerline point to an inner bronchial wall,
an inner diameter based upon the inner radius,
an outer radius from the centerline point to an outer bronchial wall,
an outer diameter based upon the outer radius,
an artery radius of an accompanying artery,
an artery diameter based upon the artery radius,
wherein the centerline point comprises a point on a centerline of the extracted centerlines.

4. Method according to claim 3, comprising determining for the at least one centerline point at least one of
a first difference between the outer radius and the inner radius as a function of the lumen diameter,
a second difference between the inner bronchial diameter and the artery diameter as a function of the lumen diameter

5. Method according to claim 1, comprising displaying an indicator indicating a position in the tracheobronchial tree corresponding to the quantitative measurement

6. Method according to claim 5, wherein the indicator indicates an anomaly within the tracheobronchial tree.

7. Method according to claim 1, wherein an image acquisition device designed to reconstruct a volumetric image set acquires the three-dimensional image set.

8. Method according to claim 1, wherein the trachcobronical tree is displayed partially.

9. Method according to claim 1, wherein a user can manipulate the automatic segmentation and/or the displayed tracheobronchial tree.

10. System for displaying a tracheobronchial tree comprising segmentation means for automatically segmenting the tracheobronchial tree from a three-dimensional image set of a body.

11. System according to claim 10, comprising;
determining means D for automatically determining a quantitative measurement based upon the tracheobronchial tree;
displaying means for displaying the quantitative measurement.

12. System according to claim 10, comprising;
extracting means for extracting centerlines of trachea, bronchi, and/or smaller airways based upon the tracheobronchial tree; and
the determining means further is arranged to determine branching points of the tracheobronchial tree based upon the extracted centerlines.

13. System according to claim 12, wherein the determining means further is arranged to determine for at least one centerline point at least one of
a bronchial lumen,
a lumen diameter,
an inner radius from the centerline point to an inner bronchial wall,
an inner diameter based upon the inner radius,
an outer radius from the centerline point to an outer bronchial wall,
an outer diameter based upon the outer radius,
an artery radius of an accompanying artery,
an artery diameter based upon the artery radius,
wherein the centerline point comprises a point on a centerline of the extracted centerlines.

14. System according to claim 13, wherein the determining means further is arranged to determine for at least one branching point at least one of
a first difference between the outer radius and the inner radius as a function of the lumen diameter,
a second difference between the inner bronchial diameter and the artery diameter as a function of the lumen diameter.

15. System according to claim 11, wherein the display means further is arranged to display an indicator indicating a correspondence between the tracheobronchial tree and the quantitative measurement.

16. An image acquisition device comprising the System according to claim 11.

17. An image workstation comprising the System according to claim 11.

18. A computer program product designed to perform the method according to claim 1.

19. An information carrier comprising the computer program product according to claim 18.
